(19) United States
(12) Patent Application Publication
(10) Pub. No.: US 2026/0115388 A1
(43) Pub. Date: Apr. 30, 2026

(54) CONTINUOUS MICRO-DOSE ADMINISTRATION APPARATUS AND INJECTION METHOD

(71) Applicant: SHANGHAI XURUN MEDICAL TECHNOLOGY CO., LTD, Shanghai (CN)

(72) Inventors: Bin XI, Shanghai (CN); Weinong XI, Shanghai (CN); Mingsheng CHEN, Shanghai (CN)

(73) Assignee: SHANGHAI XURUN MEDICAL TECHNOLOGY CO., LTD, Shanghai (CN)

(21) Appl. No.: 19/430,316

(22) Filed: Dec. 23, 2025

Related U.S. Application Data
(63) Continuation of application No. PCT/CN2024/137277, filed on Dec. 6, 2024.

(30) Foreign Application Priority Data
Dec. 8, 2023 (CN) 202311685633.6

(51) Int. Cl. A61M 5/315; A61M 5/145; A61M 5/20

(52) U.S. Cl. CPC A61M 5/31593; A61M 5/20; A61M 2005/14506; A61M 2205/0272; A61M 2205/8275

(57) ABSTRACT

A continuous micro-dose administration apparatus includes a needle apparatus, a drug storage apparatus, a piston push rod apparatus, a force accumulating apparatus, an accumulated force release apparatus, and a control apparatus. Under the action of the control apparatus, the accumulated force release apparatus releases a specified piston stroke, the force accumulating apparatus pushes the piston push rod apparatus to move into the drug storage apparatus by the specified piston stroke, and drug liquid in the drug storage apparatus is discharged through the needle apparatus. With the force accumulating apparatus as a power source, the piston body is pushed by means of rotation or direct pushing, a required number of pulses is calculated according to a dose to be infused or a distance moved by the piston, and the control apparatus accurately controls the accumulated force release apparatus to release the specified piston stroke, thereby improving the dose administration accuracy.

### CROSS REFERENCE TO THE RELATED APPLICATIONS

This application is the continuation application of International Application No. PCT/CN2024/137277, filed on Dec. 6, 2024, which is based upon and claims priority to Chinese Patent Application No. 202311685633.6, filed on Dec. 8, 2023, the entire contents of which are incorporated herein by reference.

### TECHNICAL FIELD

The present disclosure relates to the technical field of medical devices, and in particular to a continuous micro-dose administration apparatus and an injection method.

### BACKGROUND

Continuous administration refers to continuously administering a drug every day to maintain an effective blood drug concentration. According to existing clinical medical systems, continuous administration is frequently used in medical fields such as endocrinology and analgesia.

Taking the treatment for diabetes as an example, there are mainly two methods for treatment and control of diabetes at present, i.e., the conventional treatment method and the intensive treatment method. The former involves 1-2 daily insulin injections and 1-2 daily blood glucose monitoring sessions. The latter involves frequent daily blood glucose monitoring, and continuous insulin infusion that simulates the insulin secretion of pancreatic islet cells, so as to keep the blood glucose as close to normal levels as possible, and this method is mostly accomplished with the use of an insulin pump.

In 1993, the above two methods were used by the Diabetes Control and Complications Trial (DCCT) to conduct the largest long-term clinical trial to investigate the benefits of intensive blood glucose control. Results reveal that the average glycated hemoglobin in the well-controlled group was decreased by 2% in comparison with the poorly controlled group, greatly reducing the risk of chronic diabetic complications. Specifically, the risk of diabetic ocular complications was reduced by approximately 76%, and the risk of renal and neurological complications was reduced to a similar extent.

The Chinese Patent Application No. CN113633850B provides an automatic insulin injection device, including an insulin pen. A protective sleeve configured to protect a needle is connected to a needle end of the insulin pen. A connecting device configured to realize automatic injection of the insulin pen and allow the needle to penetrate into the patient's skin is disposed between an injection push button of the insulin pen and the protective sleeve. This automatic insulin injection device realizes insertion of the needle into the subcutaneous fat of the patient and automatically triggers the infusion of the insulin dose.

The automatic insulin injection device in the prior art has difficulty in accurately controlling administered doses, complex structure, and high manufacturing cost, and thus needs to be improved.

### SUMMARY

In view of defects in the prior art, an objective of the present disclosure is to provide a continuous micro-dose administration apparatus and an injection method.

The present disclosure provides a continuous micro-dose administration apparatus, including a needle apparatus, a drug storage apparatus, a piston push rod apparatus, a force accumulating apparatus, an accumulated force release apparatus, and a control apparatus, where under the action of the control apparatus, the accumulated force release apparatus releases a specified piston stroke, the force accumulating apparatus pushes the piston push rod apparatus to move into the drug storage apparatus by the specified piston stroke, and drug liquid in the drug storage apparatus is discharged through the needle apparatus.

Preferably, the accumulated force release apparatus includes a rotary magnetic brake assembly and a coupling assembly; the rotary magnetic brake assembly is electrically connected to the control apparatus; the rotary magnetic brake assembly is configured to control a piston stroke of the piston push rod apparatus through the coupling assembly; and the force accumulating apparatus is configured to output a rotational torque or a linear thrust to the piston push rod apparatus.

Preferably, the rotary magnetic brake assembly includes a fixed coil and a permanent magnet that is rotatably provided; the permanent magnet is radially magnetized; a magnetic field generated by the fixed coil interacts with a radial magnetic field of the permanent magnet to push the permanent magnet to rotate or locate; an output gear is fixedly disposed on the permanent magnet; the force accumulating apparatus includes an elastic member, and the elastic member is in a compressed state; the coupling assembly includes a gear set; the output gear is engaged with a transmission starting gear of the gear set; a winding wheel is disposed on a transmission ending gear of the gear set; a pull wire is wound on the winding wheel; and an end of the pull wire away from the piston push rod apparatus via the elastic member extends along a telescopic direction of the elastic member to the piston push rod apparatus and is connected to the piston push rod apparatus.

Preferably, the rotary magnetic brake assembly includes a fixed coil and a permanent magnet that is rotatably provided; the permanent magnet is radially magnetized; a magnetic field generated by the fixed coil interacts with a radial magnetic field of the permanent magnet to push the permanent magnet to rotate or locate; an output gear is fixedly disposed on the permanent magnet; the force accumulating apparatus includes a first torsional spring, and the first torsional spring is in a compressed state; the coupling assembly includes a gear set; the output gear is engaged with a transmission starting gear of the gear set; a worm is disposed on a transmission ending gear of the gear set; the piston push rod apparatus includes a piston body, a lead screw, a sleeve assembly, and a worm wheel; the piston body is disposed in the drug storage apparatus; one end of the lead screw is fixedly connected to the piston body; the other end of the lead screw extends into the sleeve assembly and is threadedly connected to an inner wall of the sleeve assembly; the worm wheel is fixedly connected to an outer wall of the sleeve assembly; the worm wheel is engaged with the worm; and one end of the first torsional spring is fixedly connected to the sleeve assembly, and the other end of the first torsional spring is fixedly connected to a housing of an injection apparatus.

Preferably, the sleeve assembly includes a lead screw sleeve, a worm wheel sleeve, and a locking assembly; the lead screw sleeve extends into the worm wheel sleeve; an outer wall of the lead screw sleeve is in a sliding fit with an inner wall of the worm wheel sleeve along an axial direction of the sleeve assembly; the locking assembly is configured to fixedly connect the lead screw sleeve and the worm wheel sleeve; the lead screw extends into the lead screw sleeve; and the worm wheel is disposed on an outer wall of the worm wheel sleeve.

Preferably, the locking assembly includes a locking steel ball and a locking groove; the locking groove is relatively fixedly connected to the worm wheel sleeve; the lead screw sleeve passes through the locking groove and extends into the worm wheel sleeve; the locking steel ball is disposed on an outer surface of the lead screw sleeve; an accommodating groove with a gradually decreasing diameter is disposed on a side of the locking groove adjacent to the locking steel ball; and the locking steel ball is in an embedding fit with the accommodating groove.

Preferably, a safety valve is disposed on a connecting tube between the drug storage apparatus and the needle apparatus; the safety valve includes a valve body, a diaphragm, and a sealing ring; a liquid outlet is formed in the valve body; the liquid outlet communicates with the needle apparatus; the sealing ring and the diaphragm are disposed on the valve body in sequence; the liquid outlet is located in a middle of the sealing ring; a side of the diaphragm away from the sealing ring communicates with the drug storage apparatus; a liquid inlet is formed in the diaphragm; a flow limiting hole is formed in the sealing ring; a flow guiding groove is formed in the valve body; the liquid inlet communicates with the flow limiting hole, and the flow limiting hole communicates with the flow guiding groove; when the safety valve is open, a liquid discharging cavity is formed between the diaphragm and the liquid outlet, and the flow guiding groove communicates with the liquid discharging cavity; and when the safety valve is closed, the diaphragm is attached to the liquid outlet.

Preferably, the needle apparatus includes a vertical needle hub, a rotary needle hub, a hard needle hub, a soft needle hub, a hard needle, and a soft needle; the rotary needle hub is disposed in the vertical needle hub in a rotatable and liftable manner; a rotary needle hub guiding groove is formed in an outer wall of the rotary needle hub; the rotary needle hub guiding groove spirally extends downward from an upper end of the rotary needle hub to a lower end of the rotary needle hub, and then spirally extends upward from the lower end of the rotary needle hub to the upper end of the rotary needle hub; a vertical needle hub guiding groove is vertically formed in an inner wall of the vertical needle hub; both the soft needle hub and the hard needle hub are slidably disposed in the vertical needle hub guiding groove in sequence from bottom to top; the soft needle is connected to an underside of the soft needle hub; the hard needle is connected to an underside of the hard needle hub; the hard needle passes through the soft needle hub and the soft needle in sequence from top to bottom; and the hard needle hub is slidably disposed in the rotary needle hub guiding groove.

An injection method of the continuous micro-dose administration apparatus of the present disclosure is provided, including following steps:
step S1: filling the drug storage apparatus with a sufficient amount of drug liquid;
step S2: acquiring, by the control apparatus, an externally input injection instruction or injection program; and
step S3: controlling, by the control apparatus, the accumulated force release apparatus to release the specified piston stroke; and pushing, by the force accumulating apparatus, the piston push rod apparatus to move into the drug storage apparatus by the specified piston stroke, thereby completing quantitative injection.

Preferably, the step S3 includes:
step S3.1: converting, by the control apparatus, the externally input injection instruction or injection program into a series of corresponding electric pulse signals, and transmitting the series of corresponding electric pulse signals to the fixed coil;
step S3.2: driving, by the fixed coil, the permanent magnet to move correspondingly, such that the output gear rotates, and thereby drives the worm wheel via the gear set to rotate, where the lead screw sleeve moves into the worm wheel sleeve, until the locking steel ball is embedded into the accommodating groove of the locking groove to lock the lead screw sleeve and the worm wheel sleeve; and
step S3.3: acquiring, by the control apparatus, a locking signal for the lead screw sleeve and the worm wheel sleeve, and releasing the piston stroke contained in the externally input injection instruction via the coil, the output gear and the gear set, thereby completing the quantitative injection.

Compared with the prior art, the present disclosure has the following beneficial effects:
1. With the force accumulating apparatus as a power source, the present disclosure pushes the piston body by means of rotation or direct pushing, calculates a required number of pulses according to a dose to be infused or a distance moved by the piston, and makes use of the control apparatus to accurately control the accumulated force release apparatus to release the specified piston stroke, thereby improving the dose administration accuracy.
2. The rotary magnetic brake assembly and the piston push rod apparatus are connected through the coupling assembly formed by the gear set consisting of a plurality of gears, so the present disclosure controls a large force of the force accumulating apparatus with a small force of the rotary magnetic brake assembly.
3. When the drug liquid is filled into the drug storage cavity, the lead screw and the lead screw sleeve can move into the worm wheel sleeve through the sleeve assembly. During injection, as the worm wheel rotates, the lead screw sleeve and the worm wheel sleeve are locked by the locking assembly. The present disclosure solves the problem that the worm wheel sleeve and the lead screw sleeve need to slide freely during drug filling but lock each other during injection.
4. According to the present disclosure, with the safety valve, in case of a coupling failure, a pressure on a side of the diaphragm close to the drug storage apparatus increases suddenly under the limitation of the flow limiting hole. As a result, the diaphragm deforms toward the liquid outlet, and the diaphragm is attached to an arc-shaped sealing surface of a sealing boss structure to cover the liquid outlet, such that the liquid outlet is sealed, and the drug liquid cannot flow out, thereby solving the safety problem in the failure of the equipment.

### BRIEF DESCRIPTION OF THE DRAWINGS

Other features, objectives, and advantages of the present disclosure will become more apparent by reading the detailed description of non-limiting embodiments with reference to the following accompanying drawings.

FIG. 1 is a schematic view of an overall structure of an injection apparatus according to the present disclosure;

FIG. 2 is a schematic exploded view of an overall structure of a rotary magnetic brake assembly according to the present disclosure;

FIG. 3 is a schematic view of an overall structure of a rotary magnetic brake assembly according to the present disclosure;

FIG. 4 is a schematic view of a cooperation structure between a coupling assembly and a force accumulating apparatus according to Embodiment 1 of the present disclosure;

FIG. 5 is a schematic exploded view of an overall structure of a needle apparatus according to the present disclosure;

FIG. 6 is a schematic structural view of a needle hub of a needle apparatus according to the present disclosure;

FIG. 7 is an exploded view of an overall structure of a safety valve according to the present disclosure;

FIG. 8 is a sectional view of an overall structure of a safety valve according to the present disclosure;

FIG. 9 is a schematic exploded view of an overall structure of an injection apparatus according to Embodiment 2 of the present disclosure;

FIG. 10 is a schematic exploded view of an overall structure of a locking assembly according to the present disclosure; and

FIG. 11 is a sectional view of an overall structure of a piston push rod apparatus according to the present disclosure.

Reference numerals:

| | | |
|---|---|---|
| 1: accumulated force | 32: lead screw | 57: rotary needle |
| release apparatus | | hub guiding groove |
| 11: rotary magnetic | 33: worm wheel | 58: vertical needle |
| brake assembly | | hub guiding groove |
| 111: fixed coil | 34: sleeve assembly | 59: second torsional |
| | | spring |
| 112: permanent magnet | 341: lead screw | 510: needle insertion |
| | sleeve | button |
| 113: output gear | 342: worm wheel | 6: control apparatus |
| | sleeve | |
| 114: magnetic conductive | 35: locking assembly | 61: circuit board |
| sheet | | |
| 115: limiting hole | 351: locking steel ball | 7: safety valve |
| 12: coupling assembly | 352: locking groove | 71: valve body |
| 121: gear set | 353: accommodating | 72: diaphragm |
| | groove | |
| 122: transmission | 354: locking trigger | 721: liquid inlet |
| starting gear | reed | |
| 123: transmission | 36: guiding rod | 73: sealing ring |
| ending gear | | |
| 124: winding wheel | 4: drug storage | 731: flow limiting |
| | apparatus | hole |
| 125: pull wire | 41: drug storage cavity | 74: liquid outlet |
| 126: worm | 5: needle apparatus | 75: flow guiding |
| | | groove |
| 127: guiding post | 51: vertical needle hub | 76: annular |
| | | accommodating |
| | | groove |
| 2: force accumulating | 52: rotary needle hub | 77: sealing boss |
| apparatus | | structure |
| 21: compression spring | 53: hard needle hub | 8: housing |
| 22: first torsional | 54: soft needle hub | 81: sleeve locking |
| spring | | signal switch |
| 3: piston push rod | 55: hard needle | 82: guiding seat |
| apparatus | | |
| 31: piston body | 56: soft needle | |

### DETAILED DESCRIPTION OF THE EMBODIMENTS

The present disclosure is described in detail below with reference to specific embodiments. The following embodiments will help those skilled in the art to further understand the present disclosure, but do not limit the present disclosure in any way. It should be noted that several variations and improvements can also be made by a person of ordinary skill in the art without departing from the conception of the present disclosure. These all fall within the protection scope of the present disclosure.

### Embodiment 1

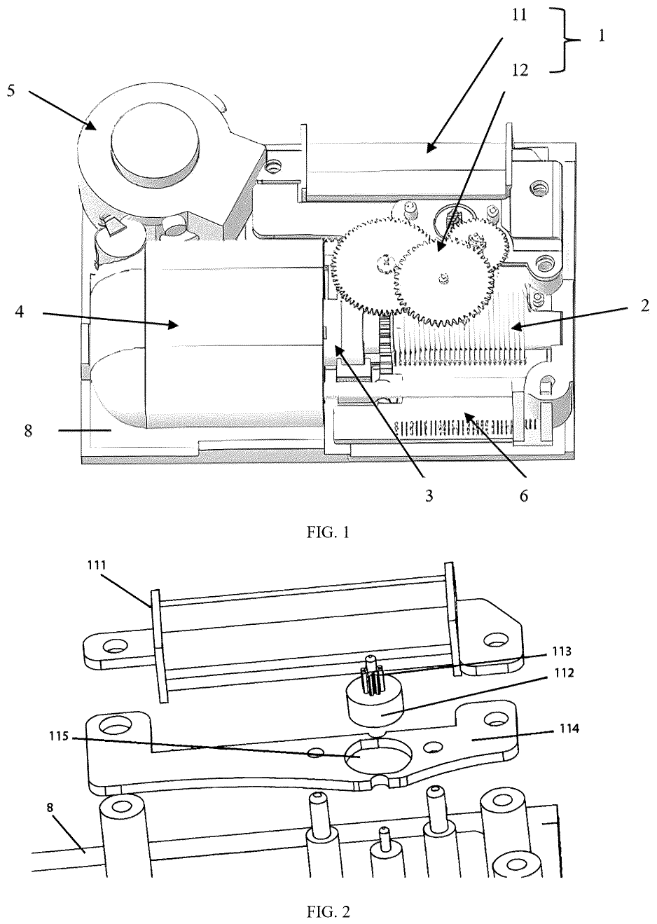

As shown in FIG. 1, the present disclosure provides a continuous micro-dose administration apparatus, including needle apparatus 5, drug storage apparatus 4, piston push rod apparatus 3, force accumulating apparatus 2, accumulated force release apparatus 1, and control apparatus 6. Under the action of the control apparatus 6, the accumulated force release apparatus 1 releases a specified piston stroke, the force accumulating apparatus 2 pushes the piston push rod apparatus 3 to move into the drug storage apparatus 4 by the specified piston stroke, and drug liquid in the drug storage apparatus 4 is discharged through the needle apparatus 5.

In the present disclosure, an operator sends an infusion instruction to the control apparatus 6 by means of an information transmission method such as Bluetooth communication, and the control apparatus 6 sends a corresponding electrical pulse to the accumulated force release apparatus 1 upon receiving the infusion instruction, realizing release of the specified piston stroke. The force accumulating apparatus 2 can push the piston push rod apparatus 3 to move into the drug storage apparatus 4 by the specified piston stroke, thereby squeezing the drug liquid in the drug storage apparatus 4 and discharging the squeezed drug liquid through a needle of the needle apparatus 5 via a tube.

Specifically, the injection apparatus further includes housing 8. The housing 8 is configured as a mounting base for components on the injection apparatus, and has a certain structural strength. The accumulated force release apparatus 1 includes rotary magnetic brake assembly 11 and coupling assembly 12. The rotary magnetic brake assembly 11 is electrically connected to the control apparatus 6. The rotary magnetic brake assembly 11 is configured to control a piston stroke of the piston push rod apparatus 3 through the coupling assembly 12. The force accumulating apparatus 2 is configured to output a linear thrust to the piston push rod apparatus 3.

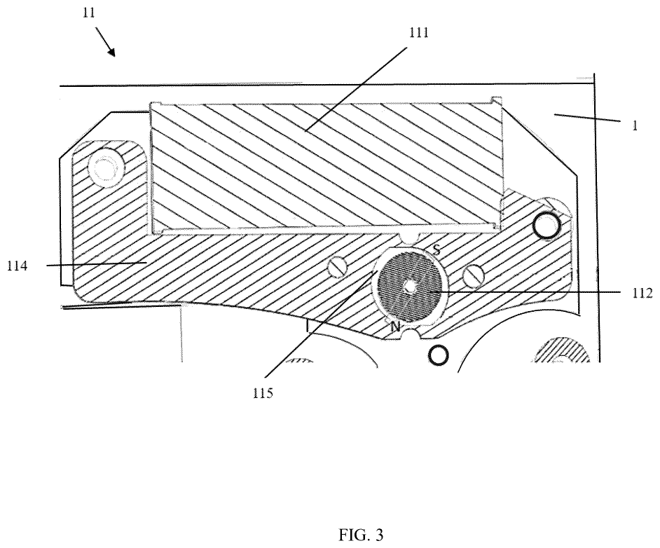

As shown in FIG. 1, FIG. 2, and FIG. 3, more specifically, the rotary magnetic brake assembly 11 includes fixed coil 111 and permanent magnet 112 that is rotatably provided. The permanent magnet 112 is radially magnetized. A magnetic field generated by the fixed coil 111 interacts with a radial magnetic field of the permanent magnet 112 to push the permanent magnet 112 to rotate or locate. Output gear 113 is fixedly disposed on the permanent magnet 112. The fixed coil 111 includes a coil and a fixing bracket. The coil is wound on the fixing bracket. The fixing bracket is fixedly disposed on the housing 8 through a fastener. Magnetic conductive sheet 114 is further fixedly disposed on the housing 8 through a fastener. Limiting hole 115 configured to allow the permanent magnet 112 to rotate and locate is formed in the magnetic conductive sheet 114. The permanent magnet 112 is cylindrical. The cylindrical permanent magnet 112 is disposed in the limiting hole 115 and rotatably disposed on the housing 8 through a coaxially disposed rotating shaft. The magnetic conductive sheet 114 can be configured to generate a magnetic field with an alternating direction. The magnetic field generated by the fixed coil 111 can interact with the magnetic field of the permanent magnet 112, causing the permanent magnet 112 to rotate by a certain angle, particularly by 180°. The output gear 113 is coaxially and fixedly disposed on the permanent magnet 112. As the permanent magnet 112 rotates, the output gear 113 can be driven to rotate.

It is to be noted that the rotary magnetic brake assembly 11 in the present disclosure can further be an electromagnetic brake, an electromagnetic swinging fork or an electromagnetic ring capable of outputting a torque in the prior art.

The control apparatus 6 includes circuit board 61. A communication module, a data processing module, a data storage module, and a signal conversion module are integrated onto the circuit board 61. The signal conversion module is electrically connected to the communication module. The circuit board 61 is electrically connected to the fixed coil 111. The communication module of the control apparatus 6 can be configured to receive an external wired or wireless infusion instruction signal or infusion program. According to received infusion information, the data processing module can be configured to automatically control the signal conversion module to generate a corresponding electrical pulse signal as needed. The electrical pulse signal is transmitted to the fixed coil 111 to control the permanent magnet 112 to rotate by a certain angle, thereby controlling the output gear 113 to rotate by a certain angle.

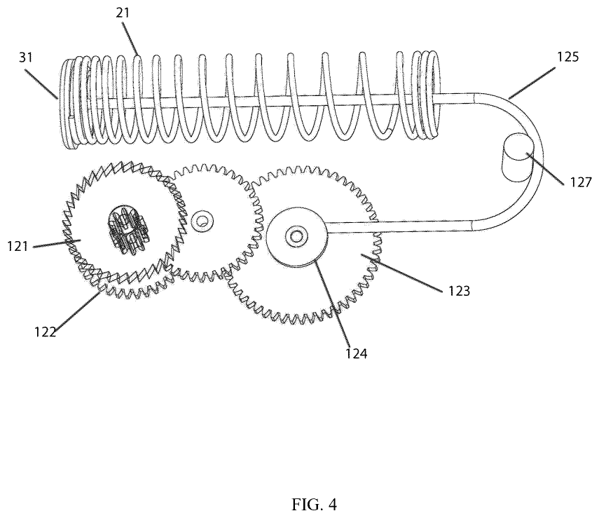

As shown in FIG. 1, FIG. 2, FIG. 3, and FIG. 4, the coupling assembly 12 includes gear set 121. The gear set 121 includes a plurality of engaged gear structures. Any gear of the gear set 121 is rotatably connected to the housing 8 through a rotating shaft. The gear set 121 includes at least one transmission starting gear 122 and at least one transmission ending gear 123. The output gear 113 is engaged with the transmission starting gear 122 of the gear set 121. At least one intermediate transmission gear is disposed between the transmission starting gear 122 and the transmission ending gear 123. The intermediate transmission gear is engaged with the transmission starting gear 122 and another intermediate transmission gear, or engaged with the transmission ending gear 123 and another intermediate transmission gear, or engaged with another two intermediate transmission gears.

The force accumulating apparatus 2 includes an elastic member, and the elastic member is in a compressed state. In the present disclosure, the elastic member is configured to output a linear thrust, and may be an elastic apparatus capable of storing potential energy in the prior art, such as compression spring 21, a rubber band, or a compressed air spring. The elastic member in the embodiment of the present disclosure is preferably the compression spring 21.

Winding wheel 124 is coaxially disposed on the transmission ending gear 123 of the gear set 121. Pull wire 125 is wound on the winding wheel 124. An end of the pull wire 125 away from the piston push rod apparatus 3 via the compression spring 21 extends along a telescopic direction of the compression spring 21 to the piston push rod apparatus 3 and is connected to the piston push rod apparatus 3. The gear set 121 can be configured to convert a rotation angle of the output gear 113 into a linear motion stroke of the pull wire 125 at a certain ratio. When the drug liquid is to be injected, a specific infusion instruction is sent by the user to the control apparatus 6. The control apparatus 6 sends a corresponding electrical pulse signal to the fixed coil 111 upon receiving the specific infusion instruction, causing the permanent magnet 112 and the output gear 113 to rotate by a certain angle. The output gear 113 releases a certain length of the pull wire 125 through the gear set 121, realizing the release of the specified piston stroke. At this time, the specified piston stroke is the same as the released length of the pull wire 125.

Further, the gear set 121 and the compression spring 21 are arranged side by side. Guiding post 127 is disposed on the housing 8. The guiding post 127 is disposed at a side of the compression spring 21 away from the piston push rod apparatus 3. The pull wire 125 on the winding wheel passes around the guiding post 127 first and then extends through an end of the compression spring 21 away from the piston push rod apparatus 3 to the piston push rod apparatus 3 along the telescopic direction of the compression spring 21 and is connected to the piston push rod apparatus 3.

The piston push rod apparatus 3 includes piston body 31. The drug storage apparatus 4 includes drug storage cavity 41. The piston body 31 extends into the drug storage cavity 41 and is slidably connected to an inner wall of the drug storage cavity 41. A movable seal is disposed between the piston body 31 and the inner wall of the drug storage cavity 41, thereby preventing leakage of the drug liquid from a junction between the piston body 31 and the inner wall of the drug storage cavity 41. A movement direction of the piston body 31 in the drug storage cavity 41 is parallel to the telescopic direction of the compression spring 21. The compression spring 21 is connected or crimped to the piston body 31. The compression spring 21 in the compressed state stores sufficient elastic potential energy. When the gear set 121 releases a certain length of the pull wire 125, the piston body 31 can move a certain length into the drug storage cavity 41 under the action of the compression spring 21. It is to be noted that a cross-sectional area of the drug storage cavity 41 can be determined during manufacturing. By enabling the piston body 31 to move the certain length into the drug storage cavity 41, a volume of the drug liquid discharged when the piston body 31 moves the certain length into the drug storage cavity 41 can be calculated. This realizes the quantitative infusion of the drug liquid and improves the infusion accuracy of the drug liquid.

With the gear set 121, a small force of the rotary magnetic brake assembly 11 can be used to control a large force of the force accumulating apparatus 2. While same clinical requirements are met, compared with a servo motor and a memory alloy, the technical solution of the present disclosure has a significantly reduced cost, with power consumption reduced to one-tenth or even one-hundredth. This can greatly reduce the cost of the medical device purchased by the patient and minimize the battery consumption and related pollution.

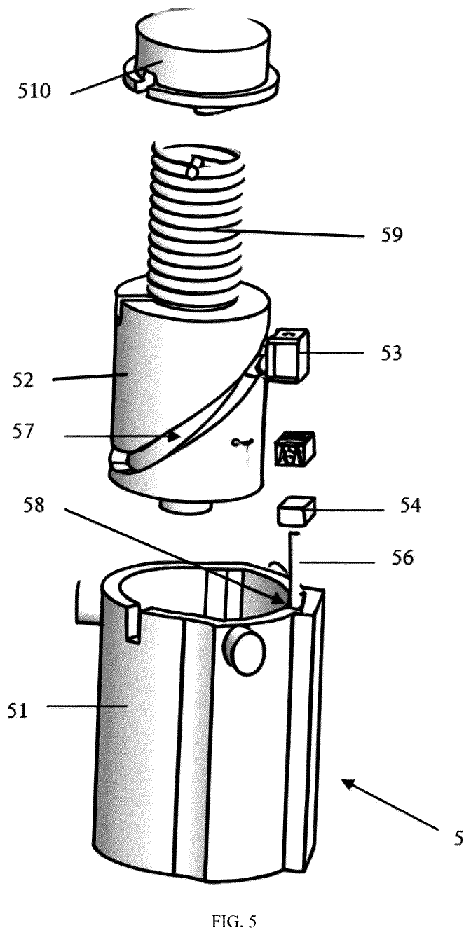

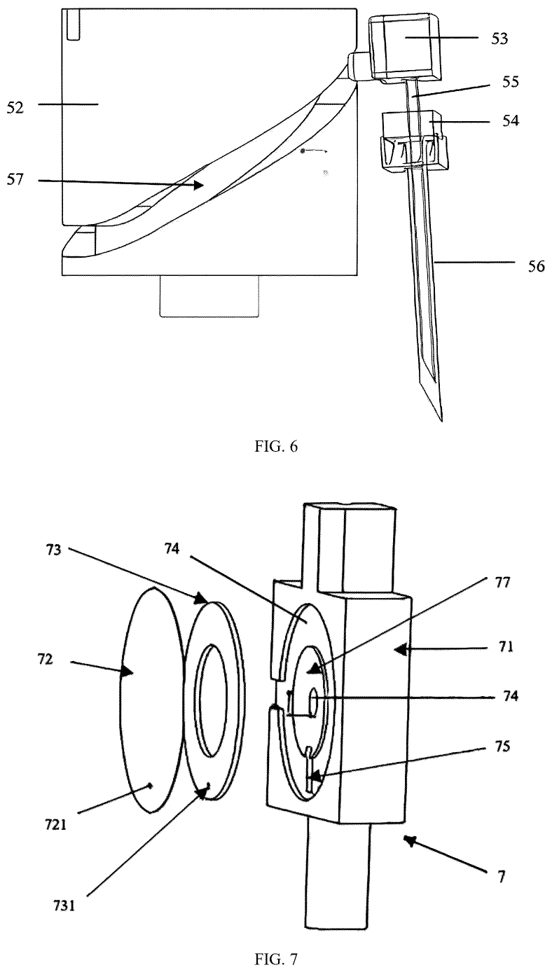

As shown in FIG. 5 and FIG. 6, the needle apparatus 5 includes vertical needle hub 51, rotary needle hub 52, hard needle hub 53, soft needle hub 54, hard needle 55, soft needle 56, second torsional spring 59, and needle insertion button 510. The vertical needle hub 51 is fixedly disposed on the housing 8. The rotary needle hub 52, the second torsional spring 59, and the needle insertion button 510 are coaxially disposed in the vertical needle hub 51 in sequence from bottom to top. The rotary needle hub 52 is disposed in the vertical needle hub 51 in a rotatable and liftable manner. The rotary needle hub 52 is in a rotating fit with an inner wall of the vertical needle hub 51. Rotary needle hub guiding groove 57 is formed in an outer wall of the rotary needle hub 52. The rotary needle hub guiding groove 57 spirally extends downward from an upper end of the rotary needle hub 52 to a lower end of the rotary needle hub 52, and then spirally extends upward from the lower end of the rotary needle hub 52 to the upper end of the rotary needle hub 52. A lower end of the second torsional spring 59 is connected or crimped to the upper end of the rotary needle hub 52. An upper end of the second torsional spring 59 is connected to the needle insertion button 510. The torsional spring under a vertical pressure can drive the rotary needle hub 52 to rotate downward along a vertical direction.

Vertical needle hub guiding groove 58 is vertically formed in the inner wall of the vertical needle hub 51. Both the soft needle hub 54 and the hard needle hub 53 are slidably disposed in the vertical needle hub guiding groove 58 in sequence from bottom to top. Both the soft needle hub 54 and the hard needle hub 53 are in a sliding fit with the vertical needle hub guiding groove 58. The soft needle 56 is connected to an underside of the soft needle hub 54. The hard needle 55 is connected to an underside of the hard needle hub 53. The hard needle 55 passes through the soft needle hub 54 and the soft needle 56 in sequence from top to bottom. The hard needle hub 53 is slidably disposed in the rotary needle hub guiding groove 57. It is to be noted that the soft needle 56 is coaxially sleeved on the hard needle 55. The hard needle 55 serves to support the soft needle 56 when piercing into skin.

By pressing the needle insertion button 510, the second torsional spring 59 pushes the rotary needle hub 52 to rotate downward. The hard needle hub 53 limited by the vertical needle hub guiding groove 58 cannot move left and right, but can only slide in the rotary needle hub guiding groove 57 and is pushed by the rotary needle hub guiding groove 57 to move up and down in the vertical needle hub guiding groove 58. When the rotary needle hub guiding groove 57 rotates a front half circle, it pushes the hard needle hub 53, the hard needle 55, the soft needle hub 54, and the soft needle 56 to move downward together, piercing the hard needle 55 and the soft needle 56 into the skin. When the rotary needle hub guiding groove 57 rotates a rear half circle, it pushes the hard needle hub 53 to move upward, pulling out the hard needle 55, and leaving the soft needle 56 in the skin.

The soft needle 56 communicates with the drug storage cavity 41 through a tube. Safety valve 7 is disposed on a connecting tube between the drug storage apparatus 4 and the needle apparatus 5. The safety valve 7 is configured to connect or block a liquid path between the drug storage apparatus 4 and the needle apparatus 5.

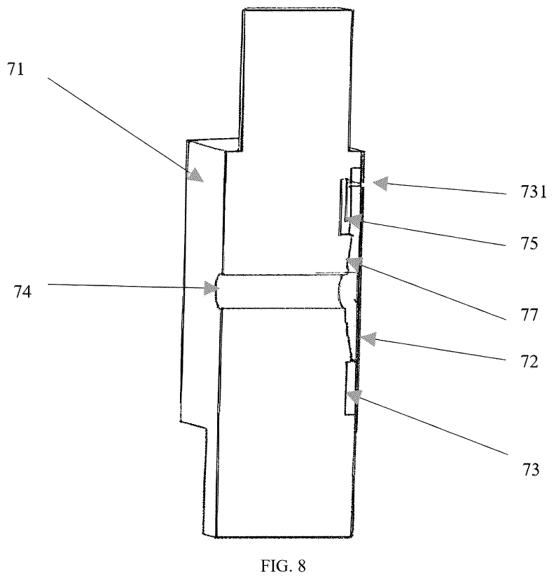

As shown in FIG. 7 and FIG. 8, the safety valve 7 includes valve body 71, diaphragm 72, and sealing ring 73. Liquid outlet 74 is formed in the valve body 71. The liquid outlet 74 communicates with the soft needle 56 of the needle apparatus 5. The sealing ring 73 and the diaphragm 72 are disposed on the valve body 71 in sequence. The liquid outlet 74 is located in a middle of the sealing ring 73. A side of the diaphragm 72 away from the sealing ring 73 communicates with the drug storage apparatus 4. Specifically, one side of the valve body 71 is provided with annular accommodating groove 76. The liquid outlet 74 is coaxial with the annular accommodating groove 76. The sealing ring 73 is embedded into the annular accommodating groove 76. The diaphragm 72 is attached to the sealing ring 73.

Liquid inlet 721 is formed in the diaphragm 72. Flow limiting hole 731 is formed in the sealing ring 73. Flow guiding groove 75 is formed in the valve body 71. The liquid inlet 721 communicates with the flow limiting hole 731, and the flow limiting hole 731 communicates with the flow guiding groove 75.

When the safety valve 7 is open, a liquid discharging cavity is formed between the diaphragm 72 and the liquid outlet 74, and the flow guiding groove 75 communicates with the liquid discharging cavity. The drug liquid in the drug storage apparatus 4 passes through the liquid inlet 721 of the diaphragm 72 and the flow limiting hole 731 of the sealing ring 73 in sequence, and flows to the flow guiding groove 75. A middle of the annular accommodating groove 76 in the valve body 71 is sealing boss structure 77. The sealing boss structure 77 is circular. The sealing boss structure 77 is gradually recessed from a circumferential edge to a middle to form an arc-shaped sealing surface. A gap between the arc-shaped sealing surface of the sealing boss structure 77 and the diaphragm 72 serves as the liquid discharging cavity. The flow guiding groove 75 extends from the annular accommodating groove 76 into the sealing boss structure 77. The drug liquid in the drug storage apparatus 4 passes through the liquid inlet 721, the flow limiting hole 731, and the flow guiding groove 75 to enter the liquid discharging cavity, and then enters the needle apparatus 5 from the liquid discharging cavity via the liquid outlet 74, thereby accomplishing injection of the drug liquid.

In case of a coupling failure, as the piston body 31 is pushed rapidly, a pressure on a side of the diaphragm 72 close to the drug storage apparatus 4 suddenly increases under the action of the liquid inlet 721 and the flow limiting hole 731. As a result, the diaphragm 72 deforms toward the liquid outlet 74 until the diaphragm 72 is completely attached to the arc-shaped sealing surface of the sealing boss structure 77. At this time, the diaphragm 72 is attached to the liquid outlet 74 to completely cover the liquid outlet 74, and the safety valve 7 is closed, such that the drug liquid cannot flow out.

Therefore, when the coupling assembly 12 fails, it suddenly loses the limitation on the force accumulating mechanism. The piston body 31 is pushed rapidly, and under the limitation of the flow limiting hole 731, the pressure on the side of the diaphragm 72 close to the drug storage apparatus 4 increases suddenly. As a result, the diaphragm 72 deforms toward the liquid outlet 74, the diaphragm 72 is attached to the arc-shaped sealing surface of the sealing boss structure 77 to cover the liquid outlet 74, and the liquid outlet 74 is sealed, such that the drug liquid cannot flow out. With the structure of the diaphragm safety valve 7, the safety problem in the failure of the equipment is solved.

The present disclosure provides an injection method of a continuous micro-dose administration apparatus, including following steps:
Step S1: The drug storage apparatus 4 is filled with a sufficient amount of drug liquid.
Step S2: The control apparatus 6 acquires an externally input injection instruction or injection program.
Step S3: The control apparatus 6 controls the accumulated force release apparatus 1 to release the specified piston stroke; and the force accumulating apparatus 2 pushes the piston push rod apparatus 3 to move into the drug storage apparatus 4 by the specified piston stroke, thereby completing quantitative injection.

Specifically, there is no drug liquid in the continuous micro-dose administration apparatus before use. The drug liquid is injected into the drug storage cavity 41 with a syringe. Hence, a liquid injection hole is reserved in a sidewall of the drug storage cavity 41 where an insertion stroke of a piston is terminated. When the drug liquid is injected into the drug storage cavity 41, the piston body 31 of the drug storage apparatus moves backward with injection of the drug liquid. The compression spring 21 is compressed, until the drug storage cavity 41 is filled with the sufficient amount of drug liquid. When the drug liquid is to be injected, a specific infusion instruction is sent by the user to the control apparatus 6 by means of the method such as the Bluetooth communication. The control apparatus 6 sends a corresponding electrical pulse signal to the fixed coil 111 upon receiving the specific infusion instruction, causing the permanent magnet 112 and the output gear 113 to rotate by a certain angle. The output gear 113 releases a certain length of the pull wire 125 through the gear set 121, realizing the release of the specified piston stroke. At this time, the specified piston stroke is the same as the released length of the pull wire 125. The compression spring 21 in the compressed state is restored to push the piston push rod apparatus 3 to move into the drug storage apparatus 4 by the specified piston stroke, thereby completing the quantitative injection.

### Embodiment 2

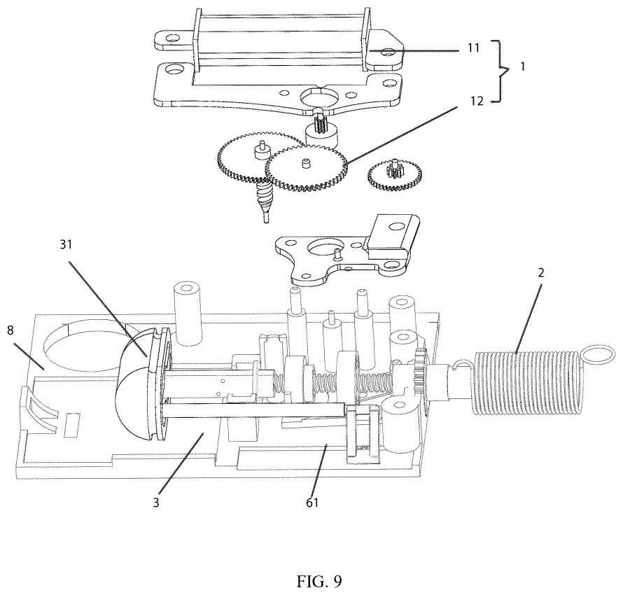

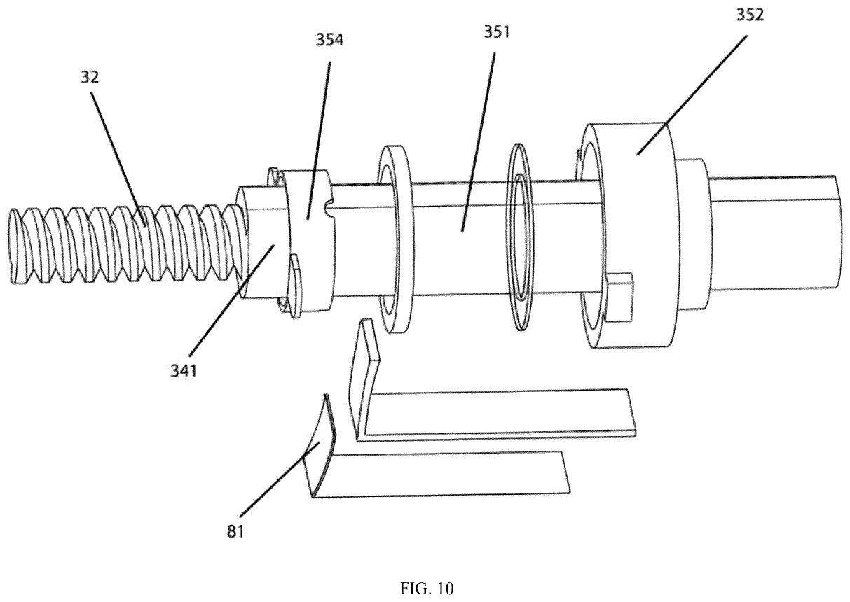

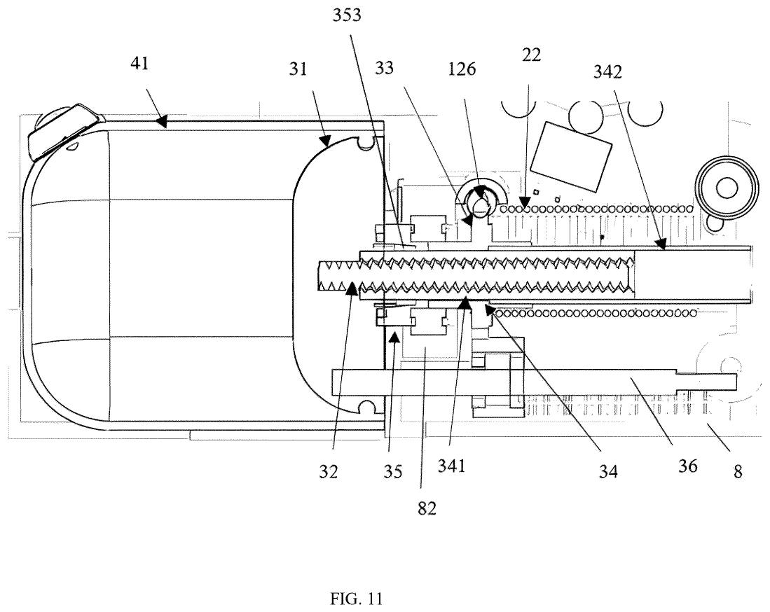

Based on Embodiment 1, as shown in FIG. 9, FIG. 10, and FIG. 11, according to the continuous micro-dose administration apparatus provided by the present disclosure, the present disclosure further provides a feasible combination of the piston push rod apparatus 3, the force accumulating apparatus 2, and the accumulated force release apparatus 1. The accumulated force release apparatus 1 includes rotary magnetic brake assembly 11 and coupling assembly 12. The rotary magnetic brake assembly 11 is electrically connected to the control apparatus 6. The rotary magnetic brake assembly 11 is configured to control the piston stroke of the piston push rod apparatus 3 through the coupling assembly 12. The force accumulating apparatus 2 is configured to output a rotational torque to the piston push rod apparatus 3.

More specifically, the rotary magnetic brake assembly 11 includes fixed coil 111 and permanent magnet 112 that is rotatably provided. The permanent magnet 112 is radially magnetized. A magnetic field generated by the fixed coil 111 interacts with a radial magnetic field of the permanent magnet 112 to push the permanent magnet 112 to rotate or locate. Output gear 113 is fixedly disposed on the permanent magnet 112. The fixed coil 111 includes a coil and a fixing bracket. The coil is wound on the fixing bracket. The fixing bracket is fixedly disposed on the housing 8 through a fastener. Magnetic conductive sheet 114 is further fixedly disposed on the housing 8 through a fastener. Limiting hole 115 configured to allow the permanent magnet 112 to rotate and locate is formed in the magnetic conductive sheet 114. Through a shape of the limiting hole 115, the permanent magnet can be located. The permanent magnet 112 is cylindrical. The cylindrical permanent magnet 112 is disposed in the limiting hole 115 and rotatably disposed on the housing 8 through a coaxially disposed rotating shaft. The magnetic conductive sheet 114 can be configured to generate a specific magnetic field, such as a magnetic field with an alternating direction. The magnetic field generated by the fixed coil 111 can interact with the magnetic field of the permanent magnet 112, causing the permanent magnet 112 to rotate by a certain angle, particularly by 180°. The output gear 113 is coaxially and fixedly disposed on the permanent magnet 112. As the permanent magnet 112 rotates, the output gear 113 can be driven to rotate.

The control apparatus 6 includes circuit board 61. A communication module, a data processing module, a data storage module, and a signal conversion module are integrated onto the circuit board 61. The signal conversion module is electrically connected to the communication module. The circuit board 61 is electrically connected to the fixed coil 111. The communication module of the control apparatus 6 can be configured to receive an external wired or wireless infusion instruction signal or infusion program. According to received infusion information, the data processing module can be configured to automatically control the signal conversion module to generate a corresponding electrical pulse signal as needed. The electrical pulse signal is transmitted to the fixed coil 111 to control the permanent magnet 112 to rotate by a certain angle, thereby controlling the output gear 113 to rotate by a certain angle.

The coupling assembly 12 includes gear set 121. The gear set 121 includes a plurality of engaged gear structures. Any gear of the gear set 121 is rotatably connected to the housing 8 through a rotating shaft. The gear set 121 includes at least one transmission starting gear 122 and at least one transmission ending gear 123. The output gear 113 is engaged with the transmission starting gear 122 of the gear set 121. Worm 126 is disposed on the transmission ending gear 123 of the gear set 121. At least one intermediate transmission gear is disposed between the transmission starting gear 122 and the transmission ending gear 123. The intermediate transmission gear is engaged with the transmission starting gear 122 and another intermediate transmission gear, or engaged with the transmission ending gear 123 and another intermediate transmission gear, or engaged with another two intermediate transmission gears.

The force accumulating apparatus 2 is configured to output the rotational torque. The force accumulating apparatus 2 includes first torsional spring 22, and the first torsional spring 22 is in a compressed state. The piston push rod apparatus 3 includes piston body 31, lead screw 32, sleeve assembly 34, and worm wheel 33. The piston body 31 is disposed in the drug storage apparatus 4. One end of the lead screw 32 is fixedly connected to the piston body 31. The other end of the lead screw 32 extends into the sleeve assembly 34 and is threadedly connected to an inner wall of the sleeve assembly 34. The worm wheel 33 is fixedly connected to an outer wall of the sleeve assembly 34. The worm wheel 33 is engaged with the worm 126. One end of the first torsional spring 22 is fixedly connected to the sleeve assembly 34, and the other end of the first torsional spring 22 is fixedly connected to the housing 8 of the injection apparatus.

Further, the sleeve assembly 34 includes lead screw sleeve 341, worm wheel sleeve 342, and locking assembly 35. The lead screw sleeve 341 extends into the worm wheel sleeve 342. An outer wall of the lead screw sleeve 341 is in a sliding fit with an inner wall of the worm wheel sleeve 342 along an axial direction of the sleeve assembly 34. The locking assembly 35 is configured to fixedly connect the lead screw sleeve 341 and the worm wheel sleeve 342. Before the lead screw sleeve 341 and the worm wheel sleeve 342 are fixedly connected by the locking assembly 35, the lead screw sleeve 341 can slide relative to the worm wheel sleeve 342 along an axis of the sleeve assembly 34. After the lead screw sleeve 341 and the worm wheel sleeve 342 are fixedly connected by the locking assembly 35, there is no relative movement between the lead screw sleeve 341 and the worm wheel sleeve 342.

The locking assembly 35 includes locking steel ball 351 and locking groove 352. The locking groove 352 is relatively fixedly connected to the worm wheel sleeve 342. The lead screw sleeve 341 passes through the locking groove 352 and extends into the worm wheel sleeve 342. The locking steel ball 351 is disposed on an outer surface of the lead screw sleeve 341. Accommodating groove 353 with a gradually decreasing diameter is disposed on a side of the locking groove 352 adjacent to the locking steel ball 351. The locking steel ball 351 is in an embedding fit with the accommodating groove 353. The accommodating groove 353 is formed by the cooperation between an inner wall of the locking groove 352 and an outer wall of the lead screw sleeve 341. The accommodating groove 353 has an approximately V-shaped section. When the locking steel ball 351 enters the locking groove 352 until a gap is less than a diameter of the locking steel ball 351, the locking steel ball 351, the locking groove 352, and the lead screw sleeve 341 seize each other, and the lead screw sleeve 341 and the worm wheel sleeve 342 lock each other.

Still further, locking trigger reed 354 is coaxially sleeved on the lead screw sleeve 341. The locking trigger reed 354 is disposed on a side of the locking steel ball 351 away from the locking groove 352. A semicircular groove in an embedding fit with the locking steel ball 351 is formed in the locking trigger reed 354. One or more locking steel balls 351 are disposed at intervals on a circumferential side of the lead screw sleeve 341 around a central axis of the lead screw sleeve 341. The semicircular groove in the locking trigger reed 354 corresponds to the locking steel ball 351. Sleeve locking signal switch 81 is disposed on the housing 8.

There is no drug liquid in the drug storage apparatus 4 before use. The drug liquid is injected into the drug storage cavity 41 with a syringe. At this time, the piston body 31 moves backward with injection of the drug liquid. The lead screw 32 and the lead screw sleeve 341 that are connected to the piston body 31 slide backward freely in the worm wheel sleeve 342. When the injection administration is started, the worm wheel sleeve 342 and the lead screw sleeve 341 must be locked and not allowed to slide freely, such that the lead screw 32 can push the piston body 31 to move forward and inject the drug liquid.

During injection, as the worm wheel starts to rotate, the locking trigger reed 354 rotates together with the worm wheel. When the sleeve locking signal switch 81 cannot block the locking trigger reed 354, the locking trigger reed 354 pushes the locking steel ball 351 into the locking groove 352. A gap between an interior of the locking groove 352 and the lead screw sleeve 341 is V-shaped. The gap between the interior of the locking groove 352 and the lead screw sleeve 341 gradually narrows. When the locking steel ball 351 enters the locking groove 352 until the gap is less than the diameter of the locking steel ball 351, the locking steel ball 351, the locking groove 352, and the lead screw sleeve 341 seize each other, and the lead screw sleeve 341 and the worm wheel sleeve 342 lock each other. This solves the problem that the worm wheel sleeve 342 and the lead screw sleeve 341 need to slide freely during drug filling but lock each other during injection.

Still further, guiding rod 36 is further fixedly disposed on the piston body 31. A length direction of the guiding rod 36 is parallel to a length direction of the lead screw 32. Guiding seat 82 is disposed on the housing 8. The guiding rod 36 horizontally passes through the guiding seat 82, and is in a sliding fit with the guiding seat 82.

The present disclosure provides an injection method of a continuous micro-dose administration apparatus, including following steps:
Step S1: The drug storage apparatus 4 is filled with a sufficient amount of drug liquid.
Step S2: The control apparatus 6 acquires an externally input injection instruction or injection program.
Step S3: The control apparatus 6 controls the accumulated force release apparatus 1 to release the specified piston stroke; and the force accumulating apparatus 2 pushes the piston push rod apparatus 3 to move into the drug storage apparatus 4 by the specified piston stroke, thereby completing quantitative injection.

Step S3 includes:
Step S3.1: The control apparatus 6 converts the externally input injection instruction or injection program into a series of corresponding electric pulse signals, and transmits the series of corresponding electric pulse signals to the fixed coil.
Step S3.2: The fixed coil 111 drives the permanent magnet 112 to move correspondingly, such that the output gear 113 rotates, and thereby drives the worm wheel 33 via the gear set 121 to rotate, where the lead screw sleeve 341 moves into the worm wheel sleeve 342, until the locking steel ball 351 is embedded into the accommodating groove 353 of the locking groove 352 to lock the lead screw sleeve 341 and the worm wheel sleeve 342.
Step S3.3: The control apparatus 6 acquires a locking signal for the lead screw sleeve 341 and the worm wheel sleeve 342, and releases the piston stroke contained in the externally input injection instruction via the coil, the output gear 113 and the gear set 121, thereby completing the quantitative injection.

During initial mounting, the accumulated force release apparatus 1, the force accumulating apparatus 2, and the piston push rod apparatus 3 are mounted in place. Since the above three components are coupled and remain stationary, the piston body is located on a top of the drug storage cavity 41. With the syringe, the insulin is filled into the drug storage cavity 41 via a filling hole. The piston body 31 is forced to move backward, triggering a power switch to turn on the circuit. Upon completion of filling, the user sends the infusion instruction through the controller by means of the Bluetooth communication. The control apparatus 6 sends the corresponding electrical pulse upon receiving the instruction. The rotary magnetic brake assembly 11 experiences angular deflection under the action of the electrical pulse, and the output gear 113 also rotates by a corresponding angle. The corresponding angle is transmitted by the coupling mechanism. The locking assembly 35 locks the lead screw sleeve 341 and the worm wheel sleeve 342, such that the force accumulating apparatus 2 can push the piston body 31 to move correspondingly. The squeezed drug liquid flows out from a tip of the soft needle 56 via the tube.

In the description of the present disclosure, it needs to be understood the orientation or positional relationships indicated by terms, such as “up”, “down”, “front”, “rear”, “left”, “right”, “vertical”, “horizontal”, “top”, “bottom”, “inside”, and “outside”, are based on the orientation or positional relationship shown in the accompanying drawings, are merely for facilitating the description of the present disclosure and simplifying the description, rather than indicating or implying that an apparatus or element referred to must have a particular orientation or be constructed and operated in a particular orientation, and therefore shall not be interpreted as limiting the present disclosure.

The specific examples of the present disclosure are described above. It should be understood that the present disclosure is not limited to the above specific implementations, and a person skilled in the art can make various variations or modifications within the scope of the claims without affecting the essence of the present disclosure. The embodiments of the present disclosure and features in the embodiments may be arbitrarily combined with each other in a non-conflicting situation.

1. A continuous micro-dose administration apparatus, comprising: a needle apparatus, a drug storage apparatus, a piston push rod apparatus, a force accumulating apparatus, an accumulated force release apparatus, and a control apparatus, wherein
under an action of the control apparatus, the accumulated force release apparatus releases a specified piston stroke, the force accumulating apparatus pushes the piston push rod apparatus to move into the drug storage apparatus by the specified piston stroke, and drug liquid in the drug storage apparatus is discharged through the needle apparatus;
the accumulated force release apparatus comprises a rotary magnetic brake assembly and a coupling assembly; the rotary magnetic brake assembly is electrically connected to the control apparatus; the rotary magnetic brake assembly is configured to control a piston stroke of the piston push rod apparatus through the coupling assembly; and the force accumulating apparatus is configured to output a rotational torque or a linear thrust to the piston push rod apparatus;
the rotary magnetic brake assembly comprises a fixed coil and a permanent magnet that is rotatably provided; the permanent magnet is radially magnetized; a magnetic field generated by the fixed coil interacts with a radial magnetic field of the permanent magnet to push the permanent magnet to rotate or locate; and an output gear is fixedly disposed on the permanent magnet;
the force accumulating apparatus comprises an elastic member, and the elastic member is in a compressed state; and
the coupling assembly comprises a gear set; the output gear is engaged with a transmission starting gear of the gear set; a winding wheel is disposed on a transmission ending gear of the gear set; a pull wire is wound on the winding wheel; and an end of the pull wire away from the piston push rod apparatus via the elastic member extends along a telescopic direction of the elastic member to the piston push rod apparatus and is connected to the piston push rod apparatus.

2. A continuous micro-dose administration apparatus, comprising: a needle apparatus, a drug storage apparatus, a piston push rod apparatus, a force accumulating apparatus, an accumulated force release apparatus, and a control apparatus, wherein
under an action of the control apparatus, the accumulated force release apparatus releases a specified piston stroke, the force accumulating apparatus pushes the piston push rod apparatus to move into the drug storage apparatus by the specified piston stroke, and drug liquid in the drug storage apparatus is discharged through the needle apparatus;
the accumulated force release apparatus comprises a rotary magnetic brake assembly and a coupling assembly; the rotary magnetic brake assembly is electrically connected to the control apparatus; the rotary magnetic brake assembly is configured to control a piston stroke of the piston push rod apparatus through the coupling assembly; and the force accumulating apparatus is configured to output a rotational torque or a linear thrust to the piston push rod apparatus;
the rotary magnetic brake assembly comprises a fixed coil and a permanent magnet that is rotatably provided; the permanent magnet is radially magnetized; a magnetic field generated by the fixed coil interacts with a radial magnetic field of the permanent magnet to push the permanent magnet to rotate or locate; and an output gear is fixedly disposed on the permanent magnet;
the force accumulating apparatus comprises a first torsional spring, and the first torsional spring is in a compressed state;
the coupling assembly comprises a gear set; the output gear is engaged with a transmission starting gear of the gear set; and a worm is disposed on a transmission ending gear of the gear set;
the piston push rod apparatus comprises a piston body, a lead screw, a sleeve assembly, and a worm wheel; the piston body is disposed in the drug storage apparatus; one end of the lead screw is fixedly connected to the piston body; the other end of the lead screw extends into the sleeve assembly and is threadedly connected to an inner wall of the sleeve assembly; the worm wheel is fixedly connected to an outer wall of the sleeve assembly; and the worm wheel is engaged with the worm; and
one end of the first torsional spring is fixedly connected to the sleeve assembly, and the other end of the first torsional spring is fixedly connected to a housing.

3. The continuous micro-dose administration apparatus according to claim 2, wherein the sleeve assembly comprises a lead screw sleeve, a worm wheel sleeve, and a locking assembly; the lead screw sleeve extends into the worm wheel sleeve; and an outer wall of the lead screw sleeve is in a sliding fit with an inner wall of the worm wheel sleeve along an axial direction of the sleeve assembly; and
the locking assembly is configured to fixedly connect the lead screw sleeve and the worm wheel sleeve; the lead screw extends into the lead screw sleeve; and the worm wheel is disposed on an outer wall of the worm wheel sleeve.

4. The continuous micro-dose administration apparatus according to claim 3, wherein the locking assembly comprises a locking steel ball and a locking groove; the locking groove is relatively fixedly connected to the worm wheel sleeve; and the lead screw sleeve passes through the locking groove and extends into the worm wheel sleeve; and
the locking steel ball is disposed on an outer surface of the lead screw sleeve; an accommodating groove with a gradually decreasing diameter is disposed on a side of the locking groove adjacent to the locking steel ball; and the locking steel ball is in an embedding fit with the accommodating groove.

5. The continuous micro-dose administration apparatus according to claim 1, wherein a safety valve is disposed on a connecting tube between the drug storage apparatus and the needle apparatus; the safety valve comprises a valve body, a diaphragm, and a sealing ring; a liquid outlet is formed in the valve body; and the liquid outlet communicates with the needle apparatus;
the sealing ring and the diaphragm are disposed on the valve body in sequence; the liquid outlet is located in a middle of the sealing ring; and a side of the diaphragm away from the sealing ring communicates with the drug storage apparatus;
a liquid inlet is formed in the diaphragm; a flow limiting hole is formed in the sealing ring; a flow guiding groove is formed in the valve body; and the liquid inlet communicates with the flow limiting hole, and the flow limiting hole communicates with the flow guiding groove;
when the safety valve is open, a liquid discharging cavity is formed between the diaphragm and the liquid outlet, and the flow guiding groove communicates with the liquid discharging cavity; and
when the safety valve is closed, the diaphragm seals the liquid outlet.

6. A continuous micro-dose administration apparatus, comprising: a needle apparatus, a drug storage apparatus, a piston push rod apparatus, a force accumulating apparatus, an accumulated force release apparatus, and a control apparatus, wherein
under an action of the control apparatus, the accumulated force release apparatus releases a specified piston stroke, the force accumulating apparatus pushes the piston push rod apparatus to move into the drug storage apparatus by the specified piston stroke, and drug liquid in the drug storage apparatus is discharged through the needle apparatus;
the needle apparatus comprises a vertical needle hub, a rotary needle hub, a hard needle hub, a soft needle hub, a hard needle, and a soft needle; and the rotary needle hub is disposed in the vertical needle hub in a rotatable and liftable manner;
a rotary needle hub guiding groove is formed in an outer wall of the rotary needle hub; and the rotary needle hub guiding groove spirally extends downward from an upper end of the rotary needle hub to a lower end of the rotary needle hub, and then spirally extends upward from the lower end of the rotary needle hub to the upper end of the rotary needle hub; and
a vertical needle hub guiding groove is vertically formed in an inner wall of the vertical needle hub; both the soft needle hub and the hard needle hub are slidably disposed in the vertical needle hub guiding groove in sequence from bottom to top; the soft needle is connected to an underside of the soft needle hub; the hard needle is connected to an underside of the hard needle hub; the hard needle passes through the soft needle hub and the soft needle in sequence from top to bottom; and the hard needle hub is slidably disposed in the rotary needle hub guiding groove.
